(19) United States
(12) Patent Application Publication
(10) Pub. No.: US 2002/0039692 A1
(43) Pub. Date: Apr. 4, 2002

(54) Photomask

(76) Inventor: Satuki Tanaka, Tokyo (JP)

Correspondence Address: ROSENMAN & COLIN LLP, 575 MADISON AVENUE, NEW YORK, NY, 10022-2585, US

(21) Appl. No.: 09/968,440

(22) Filed: Oct. 1, 2001

(30) Foreign Application Priority Data
Oct. 3, 2000 (JP) 2000-303844

(51) Int. Cl. G21G 5/00; A61N 5/00; G03C 5/00; G03G 16/00; G03F 9/00

(52) U.S. Cl. 430/5; 430/322; 430/324; 250/492.2

(57) ABSTRACT

In a photomask having a half-tone film and a light-blocking film, a half-tone region is disposed continuously between end potions of adjacent two hole patterns, and the width of the half-tone region in a direction perpendicular to a line joining the two neighboring hole patterns is established in accordance with the center-to-center distance between the neighboring two hole patterns.

### BACKGROUND OF THE INVENTION

1. Field of the Invention

The present invention relates to a photomask used in a photolithography process, and more particularly it relates to a photomask for compensating for deformation of a complex hole pattern.

2. Related Art

In a photomask used in photolithography, an important element in reducing the deformation of the hole pattern is to dispose a half-tone region between hole pattern, based on the hole pattern pitch of nearby pattern elements.

Deformation of the hole pattern can be classified into two general types. One type is deformation occurring with a large σ illumination (where a is the coherent factor, σ>0.6) or with illumination at an inclination, in which when the pitch is reduced and when the pitch is below a certain pitch, the hole diameter gradually increases. Another type of hole pattern deformation is the type occurring in which if either one of vertical pitch or horizontal pitch becomes small, there is deformation pulling in that direction.

In the past, in order to reduce the pattern deformation on a wafer, a method was used in which the mask was intentionally deformed in the reverse direction beforehand. This method is generally known as optical proximity effect correction (OPC), and there are two associated compensation methods. One method is the so-called simulation-based method, whereby a simulation in performed as required, so as to achieve the desired pattern. Another method is the rule-based method, whereby correction is performed in accordance with pre-established rules.

In the above-mentioned method, however, the prevention of hole pattern deformation becomes difficult as the hole pattern becomes finer.

Additionally, because hole pattern changes of sizes and shapes are made in units established by the design grid, if a hole pattern to be transferred large onto a wafer is corrected, there was the problem that it would become too small. This is because the mask error enhancement factor (MEEF) increases greatly as the sizes become small. The MEEF is the inclination on a graph with the horizontal axis representing the design dimension (mask dimension/reduction projection ratio) and the vertical axis representing the transfer dimension on the wafer.

Although the MEEF (inclination) is ideally 1, when the pattern becomes very small, it becomes greater than 1, and there arose the problem that the manufacturing accuracy of the mask was magnified on the wafer. In a very fine hole pattern in particular, the MEEF exceeds 4, so that even if a dimension of 10 nm is changed in the CAD data, there is a dimensional change of greater than 1 nm on the wafer. Therefore, there arose the problem in which in a case in which transferred patterns are too large, and neighboring patterns are not separated each other, if mask pattern correction is done, the hole dimension would actually become small.

In addition, in order to achieve precise control of the transfer dimension onto the wafer, it was necessary to use a finer grid in mask correction, however when using a fine grid there is an extreme increase in the amount of time required to manufacture the mask, so that this is not impractical.

There was a proposal in the past, rather than changing the mask dimension, of using inverse-phase light to change the dimension of a hole. For example, in order to improve the linearity of a hole pattern, there is a known method whereby a half-tone region is formed in an area surrounding a hole pattern. However, this method is not suitable for a complex pattern layout.

Accordingly, it is an object of the present invention to provide a photomask, whereby it is possible to correct the dimensions and deformation in a fine hole pattern and in a fine pitch thereof.

It is another object of the present invention to provide a photomask, whereby it is possible to correct dimensions and deformation of hole patterns in a complex pattern layout.

### SUMMARY OF THE INVENTION

In order to achieve the above-noted objects, the present invention adopt the following technical constitution.

Specifically, a first aspect of the present invention is a photomask comprising: a substrate which either pass or reflect light, a half-tone film formed on the substrate and having at least two hole patterns which are disposed adjacently each other with a prescribed spacing, and a light-blocking film formed on the half-tone film and having hole patterns disposed in correspondence to the hole patterns of the half-tone film and having a half-tone region provided at an area between the two hole patterns, wherein the half-tone region is continuously disposed from an end portion of one hole pattern of the hole patterns to an end portion of the other hole pattern of the hole patterns, and a width of the half-tone region along a direction perpendicular to a line joining the two hole patterns being established in accordance with a distance between centers of the two hole patterns.

A second aspect of the present invention is a photomask comprising: a substrate which either pass or reflect light, a half-tone film formed on the substrate and having at least two hole patterns which are disposed adjacently each other with a prescribed spacing, and a light-blocking film formed on the half-tone film and having hole patterns disposed in correspondence to the hole patterns of the half-tone film and having half-tone regions provided at an area between the two hole patterns, wherein one half-tone region of the half-tone regions is disposed continuously to an end portion of one hole pattern of the hole patterns and the other half-tone region of the half-tone regions is disposed continuously to an end portion of the other hole pattern of the hole patterns and the two half-tone regions are disposed opposite to each other, and a width of the half-tone region along a direction of a line joining the two hole patterns being established in accordance with a distance between centers of the two hole patterns.

In the third aspect of the present invention, a width of the half-tone region become larger as the distance between the centers of two hole patterns becomes smaller.

In the fourth aspect of the present invention, an amount of reduction in a hole dimension of the hole pattern is proportional to the width of the half-tone region and to 1/n-th power of a transmissivity of the substrate.

In the fifth aspect of the present invention, the half-tone region is provided if the distance between the centers of the two hole patterns is less than a prescribed length.

In the sixth aspect of the present invention, when a plurality of the hole patterns are arranged with high density in one dimensional direction, the half-tone region is disposed on a line joining the plurality of hole patterns in the direction.

In the seventh aspect of the present invention, when a plurality of the hole patterns are arranged with high density in two dimensional directions, the half-tone region is provided to a hole pattern disposed in an outer peripheral region of the plurality of hole patterns.

### BRIEF DESCRIPTION OF THE DRAWINGS

FIGS. 1(A) and 1(B) are drawings showing the mask pattern design process for a photomask according to the present invention.

FIG. 2(A) is a plan view showing a photomask according to the present invention.

FIG. 2(B) is a cross-sectional view along the cutting line X-X′ in FIG. 2(A).

,(A) being a plan view thereof, and (B) being.

FIG. 3 is a graph showing the relationship between pitch of the hole pattern and hole dimension for widths W of 0.06, 0.10, 0.18, and 0.30 m in the half-tone region according to the present invention.

FIG. 4 is a graph showing the relationship between pitch of the hole pattern and hole dimension as corrected according to the present invention.

FIG. 5 is a drawing showing a photomask without a half-tone region according to the comparison example 1.

FIG. 6 is a drawing showing a photomask according to the first embodiment of the present invention.

FIG. 7(A) is a drawing showing a contour line of the light intensity distribution which is the results of a simulation of the light intensity using a photomask not having a half-tone region according to the first comparison sample.

FIG. 7(B) is a drawing showing a light intensity distribution along the cutting line Y-Y′ in FIG. 7(A).

FIG. 8(A) is a drawing showing a contour line of the light intensity distribution which is the results of a simulation of the light intensity using a photomask according to the first embodiment of the present invention.

FIG. 8(B) is a drawing showing a light intensity distribution along the cutting line Z-Z′ in FIG. 8(A).

FIG. 9 is a drawing showing the mask pattern of a photomask without a half-tone region according to the comparison example 2.

FIG. 10 is a drawing showing a mask pattern of a photomask (phase-shift mask) according to the second embodiment of the present invention.

FIG. 11 is a drawing showing a contour plot of the light intensity distribution for a photomask having no mask according to the second comparison example.

FIG. 12 is a drawing showing a contour plot of the light intensity distribution for a photomask (phase-shift mask) according to the second embodiment of the present invention.

FIG. 13 is a drawing showing a mask pattern of a photomask with no half-tone region according to the third comparison example.

FIG. 14 is a drawing showing a mask pattern of a photomask according to the third embodiment of the present invention.

FIG. 15 is a drawing showing a contour plot of the light intensity distribution for a photomask having no half-tone region according to the third comparison example.

FIG. 16 is a drawing showing a contour plot of the light intensity distribution for a photomask according to the third embodiment of the present invention.

FIG. 17 is a drawing showing a mask pattern of a photomask according to the fourth embodiment of the present invention.

FIG. 18 is a drawing showing a mask pattern of a photomask without a half-tone region according to a fourth comparison example.

FIG. 19 is a drawing showing a mask pattern of a photomask according to the fourth embodiment of the present invention.

FIG. 20 is a drawing showing a contour plot of the light intensity distribution for a photomask having no half-tone region according to the fourth comparison example.

FIG. 21 is a drawing showing a contour plot of the light intensity distribution for a photomask according to the fourth embodiment.

FIG. 22(A) is a drawing showing a first mask pattern of a photomask according to the fifth embodiment of the present invention.

FIG. 22(B) is a drawing showing a second mask pattern of a photomask according to the fifth embodiment of the present invention.

FIG. 23 is a graph showing the relationship between the width W of the half-tone region and hole dimension for each pitch P for a photomask according to a fifth embodiment of the present invention.

FIG. 24 is a graph determining the optimum half-tone region width W for each pitch P for a hole dimension of 0.18 μm on a photomask according to a fifth embodiment of the present invention.

### DETAILED DESCRIPTION OF THE PREFERRED EMBODIMENTS

Embodiments of the present invention are described in detail below, with references made to relevant accompanying drawings.

(First embodiment)

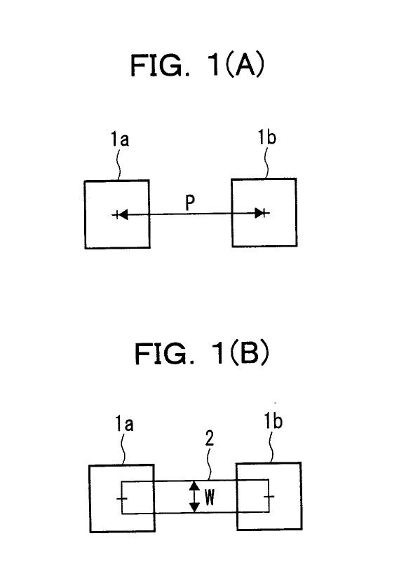

Specifically, FIG. 1 is a drawing showing the mask pattern design process for a photomask according to a first embodiment of the present invention.

As shown in FIG. 1, in the CAD data for the mask pattern, the hole patterns 1a and 1b of adjacent holes having a prescribed spacing are extracted (refer to (A)), and a rectangular (or band-shaped) half-tone pattern 2 is drawn between the hole patterns 1a and 1b (refer to (B)). The width W of the half-tone pattern 2 in a direction perpendicular to the direction of a straight line joining the neighboring hole patterns is established based on the pitch (center-to-center distance) between the hole patterns 1a and 1b.

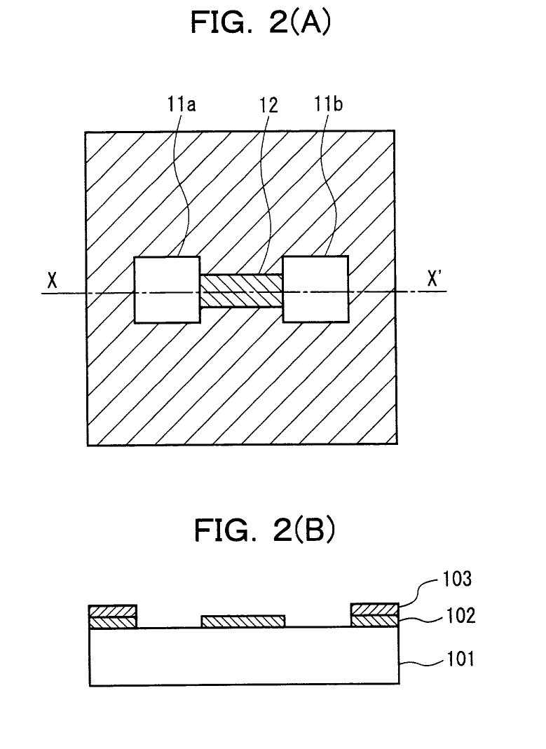

FIG. 2 is a drawing showing a photomask according to a first embodiment of the present invention, (A) being a plan view and (B) being a cross-sectional view along the cutting line X-X′. The photomask shown in FIG. 2 is fabricated by successively forming onto a transparent substrate 101 a half-tone film 102 and a light-blocking film 103. In the mask fabrication, first as shown in FIG. 1(A), the hole patterns 1a and 1b are drawn, and the light-blocking film 103 and half-tone region 102 for the hole regions 11a and 11b are etched. Next, as shown in FIG. 1(B), the half-tone pattern 2 is drawn, and the light-blocking film 103 for the half-tone region 12 is removed. At this stage, the half-tone pattern 2 in FIG. 1(B) has partial overlap with each of the hole patterns 1a and 1b, in order to achieve an overlap margin, and in order to prevent deformation of the half-tone region 2.

In the case of a long, narrow half-tone region 2, such as shown in FIG. 2, there is prominent rounding or reduction in line width at the ends or corner parts of the long, narrow half-tone region 2. For this reason, the half-tone pattern 2 is extended so that the half-tone pattern 2 extends over the hole patterns 1a and 1b, so that the rounding portions are positioned over the hole region. If this is done, in an actual mask only a part between the hole region 11a and the hole region 11b is the half-tone region 12 having the prescribed width W, the result being that the rounding or line width reduction which would have occurred at the ends or corners of the half-tone pattern 2 does not occur. The material that can be used are synthetic fused silica for the transparent substrate 101, oxide-nitride molybdenum silicide for the half-tone film 102, and chrome for the light-blocking film 103 (strictly speaking, chrome oxide film is provided to prevent reflection from the surface thereof). It is possible to use another material such as chromium fluoride or the like as the material for the half-tone film 102 for a phase-shift type photomask, in which case a different material is used for the light-blocking film 103.

Because in the half-tone region 12, the phase of the light passing through the half-tone region 12 is 180 degrees shifted from that of the hole patterns 11a and 11b, it is possible to reduce the intensity of the light in the half-tone region 12 by the interference between light having different phases. The effect of reducing the intensity of the light in the half-tone region 12 changes in accordance with the transmissivity and surface area of the half-tone region 12, the higher the transmissivity being, and the larger the surface area being, the greater is the reduction in light intensity. Therefore, by appropriately setting the transmissivity and width W of the half-tone region 12, it is possible to solve the problem of the hole pattern regions 11a and 11b being too large on the wafer, and furthermore in an extreme case there is loss of separation between two hole pattern regions 11a and 11b.

The conditions for forming the photomask according to the embodiment of the present invention are as follows. In the description that follows, the case described is that in which a random pitch of 0.18 μm is formed with a minimum hole pattern pitch is 0.3 μm. The optical conditions set in the stepper used in this case are a wavelength of 248 nm, a numerical aperture NA of 0.68, and a ⅔ annular illumination with a maximum value of a of 0.75 (illumination wherein ⅔ of the radius is blocked at the center). In illumination at an inclination or large-σ illumination (σ>0.7), the denser the pattern becomes, the greater the proximity effect causes an increase in the pattern dimension transferred to the wafer. Therefore, in the case in which the pattern interval is not somewhat small, a half-tone pattern is placed between hole patterns and as the spacing becomes greater the width W is increased gradually.

The conditions set for fabrication of the photomask are as follows. The transmissivity of the half-tone region is first set, and then it is necessary to set the pitch and the width of the half-tone region.

The transmissivity of the half-tone region should be relatively low in order to increase the precision of correction. This is because if the transmissivity of the half-tone region is excessively high, when the width is varied there will be an excessively large change in dimension on the wafer. In an extreme case, the transmissivity can be made 100%. In this case, however, because of merely providing a very fine half-tone pattern, the dimensions of the hole pattern become greatly small, so that it is not possible to achieve the object of the present invention, which is the correction of a fine hole pattern dimension or deformation. On the other hand, if transmissivity of the half-tone region is excessively low, even if the width is made maximum, it is not possible to achieve the desired reduction in dimension. For these reasons, there is an appropriate range for the transmissivity of the half-tone region. In a hole pattern having a minimum pitch of 0.3 μm and the half-tone width set to a maximum of 0.3 μm, the transmissivity of the half-tone region is set so that the size of hole pattern having a minimum pitch of 0.3 μm is same size of the independent hole. In the case of a transmissivity of 3%, the transferred dimension of the hole pattern with 0.3-μm pitch in the half-tone region is the same dimension of the independent hole in the light-blocking region. Thus, the half-tone transmissivity is set to 3%.

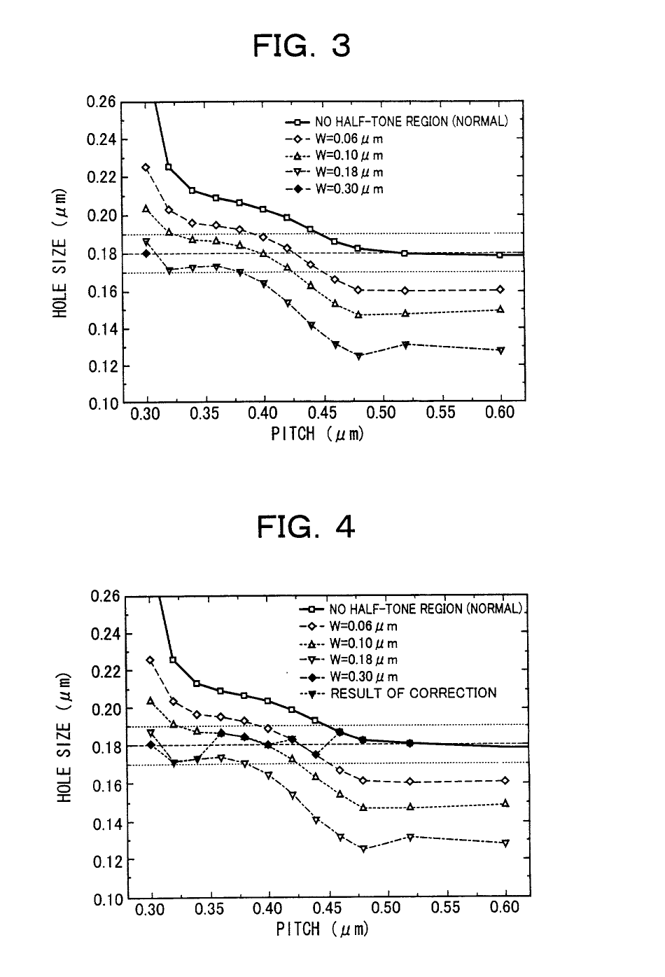

The method of setting the width of the half-tone region is as follows. The simplest method is to determine the relationship between the pitch of the hole pattern and hole dimension, and to select a half-tone region width so that the desired dimensional range is achieved for each pitch. With the half-tone region width W made 0.06, 0.10, 0.18, and 0.30 μm, the relationship between the pitch of the hole pattern and the hole dimension is as shown in FIG. 3. FIG. 3 is a graphs that shows the relationship between the pitch of the hole pattern and the hole dimension for a half-tone region width W of 0.06, 0.10, 0.18, and 0.30 μm in a photomask according to an embodiment of the present invention. The calculation was done with equal pitch along the vertical and horizontal axes. In a calculation of the light intensity distribution, the hole dimension on the wafer was determined using the so-called exposure threshold model. The light intensity used as the threshold value was the value (light intensity of approximately 0.15) which resulted in independent pattern elements (1.5-μm pitch pattern) of 0.18 μm. Considering the defocusing effect within the light-blocking film for the case of an actual exposure, the defocusing was set to 0.15 μm for the light intensity calculation. At a pitch P of 0.3 μm, the hole dimensions using a half-tone region having a width W of 0.3 μm is plotted. Therefore, in the case of a transmissivity of 3%, the minimum pitch of 0.3 μm of hole pattern is obtained. From these results, in order to have the hole dimension fall within the range of 0.18 μm±0.1 μm, the relationship between the pitch P of the hole pattern and the half-tone region width W is W=0.3 μm (for P=0.3 μm), W=0.18 μm (for P=0.31 to 0.34 μm), W=0.1 μm (for P=0.35 to 0.4 μm), and W=0.06 μm (for P=0.41 to 0.44 μm). For a pitch greater than 0.45 μm, a half-tone region was not applied. FIG. 4 shows a graph showing the dimensional change, that is the relationship between the pitch P of the hole pattern and width W of the half-tone region, when correcting using the above-mentioned rule.

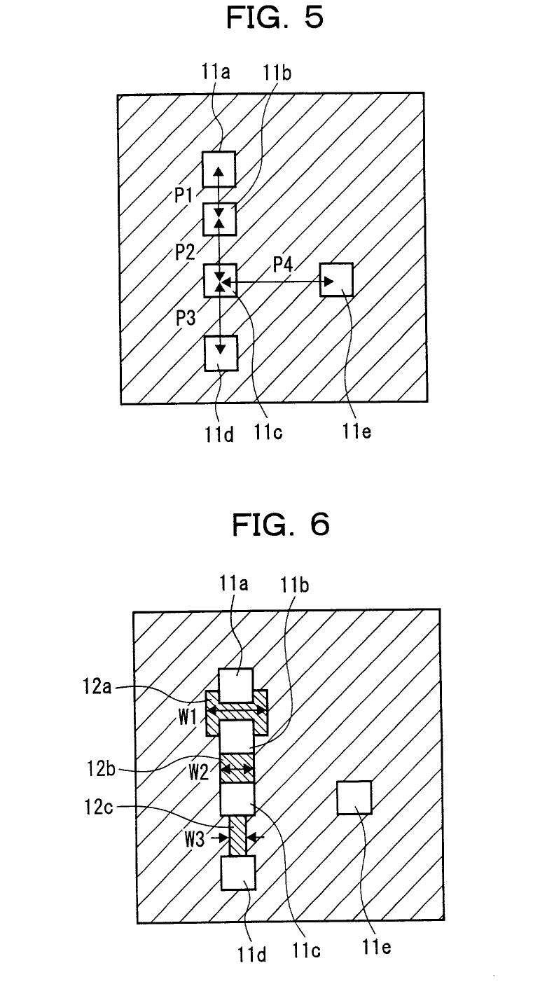

In order to verify the effect of the photomask as proposed herein, results of application to a more realistic random pattern are presented. The description thereof will use the mask shown in FIG. 5, which has 0.18 μm holes. FIG. 5 is a schematic representation showing a photomask with no half-tone region according to a first comparison example. The exposure conditions in the simulation is that an NA is 0.68, σ is 0.75 a ⅔ annular illumination (illumination wherein ⅔ of the radius is block at the center)is applied in the case of using a KrF excimer laser exposure system. The hole patterns 11a to 11d are disposed closely in the vertical direction and the hole pattern lie is somewhat distanced from the hole pattern 11c in the right horizontal direction. The pitch between the hole patterns 11a and 11b is P1, the pitch between the hole patterns 11b and 11c is P2, the pitch between hole patterns 11c and 11d is P3, and the pitch between hole patterns 11c and 11e is P4, these pitches being P1=0.3 μm, P2=0.33 μm, P3=0.44 μm, and P4=0.52 μm.

FIG. 6 shows a photomask in which a half-tone region is applied to the photomask of FIG. 5. That is, FIG. 6 is a schematic representation of a photomask according to the first embodiment of the present invention. The half-tone region 12a between the hole patterns 11a and 11b, the half-tone region 12b between the hole patterns 11b and 11c, and the half-tone region 12c between the hole patterns 11c and lid were each applied, the respective widths W1 to W3 of the half-tone regions being W1=0.3 μm, W2=0.18 μm, and W3=0.1 μm.

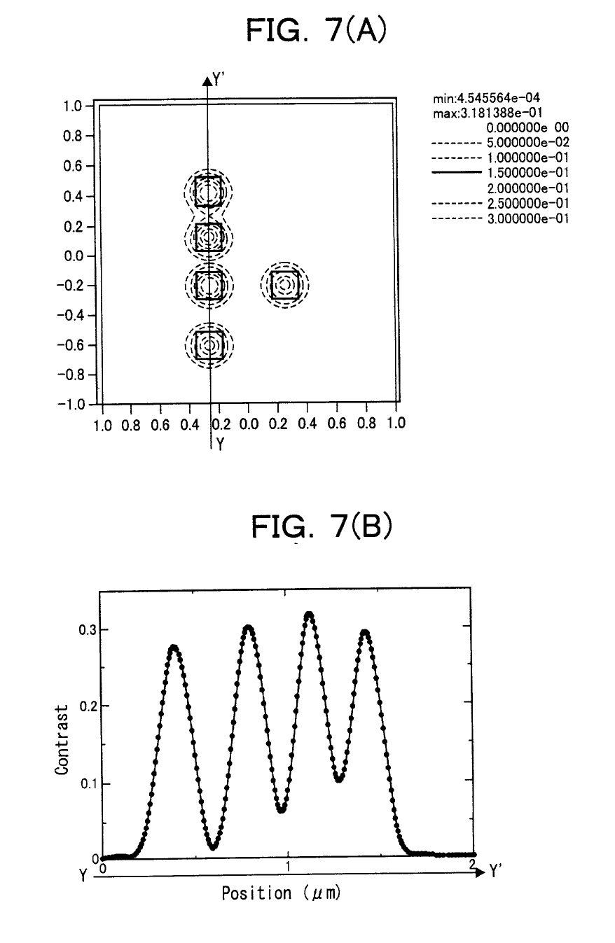

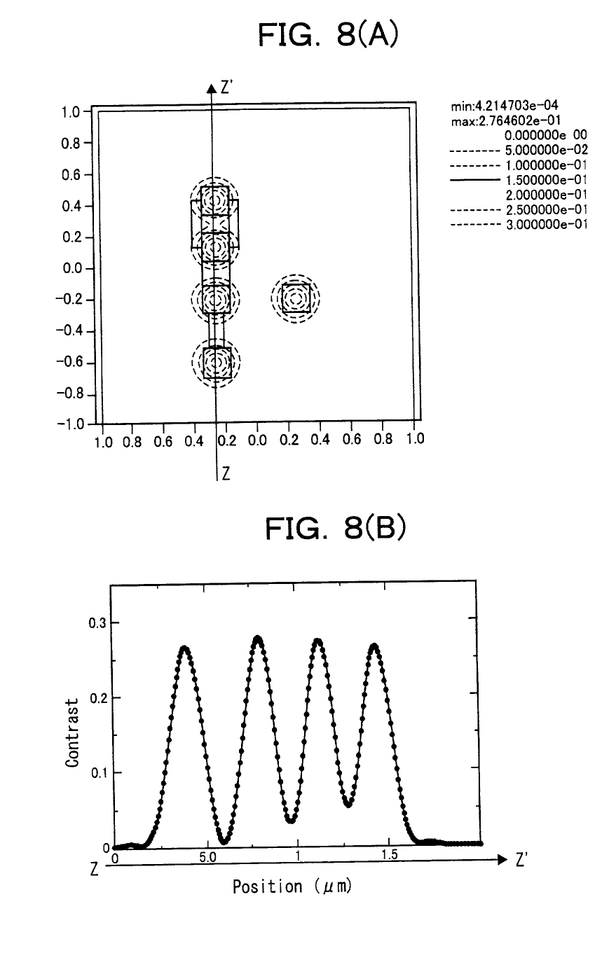

FIG. 7 shows the results of a light intensity simulation of the photomask of FIG. 5, and FIG. 8 shows the results of a light intensity simulation of the photomask of FIG. 6. FIG. 7 is a schematic representation showing the light intensity simulation results for a photomask without a half-tone region according to the first comparison example, with (A) being the contour plot of the light intensity and (B) showing contrast along the cutting line Y-Y′. FIG. 8 is a schematic representation showing the light intensity simulation results for a photomask according to the first embodiment of the present invention, with (A) being the light intensity contour plot and (B) showing contrast along the cutting line Z-Z′. Referring to FIG. 7(a) which shows the first comparison example, it can be seen that there is a deformation between pattern elements at a narrow pitch. Referring to FIG. 7(b), it can be seen that between the hole patterns 11a and 11b the minimum light intensity exceeds 0.1, so that when actual exposure is done to a wafer onto which photoresist has been applied, there will be an extremely thin film of photoresist remaining between the hole patterns 11a and 11b. Referring to FIG. 8(a) and FIG. 8(b), which show the first embodiment of the present invention, it can be seen that joining of pattern elements when the pitch is narrow is prevented.

While the foregoing description is for the case in which the transmissivity of the half-tone region is 3%, the optimum transmissivity is dependent upon the exposure condition, and on the dimensions and layout of the hole patterns. Even if the transmissivity is made somewhat high, there is no particularly great problem that occurs. The half-tone region transmissivity has a standard value for each exposure wavelength. For example, for the i-line (wave length 365 nm), this is 8%, and for a KrF exima laser (wavelength 248 nm) 6% is the generally used value (in other cases, 4% is sometimes used). Keeping a mask substrate having these standard values of transmissivity mask marker at all times is advantageous both in terms of timely completion of mask fabrication and for quality assurance purposes.

As described above, the first embodiment of the present invention is a photomask comprising: a substrate which either pass or reflect light, a half-tone film formed on the substrate and having at least two hole patterns which are disposed adjacently each other with a prescribed spacing, and a light-blocking film formed on the half-tone film and having hole patterns disposed in correspondence to the hole patterns of the half-tone film and having a half-tone region provided at an area between the two hole patterns, wherein the half-tone region is continuously disposed from an end portion of one hole pattern of the hole patterns to an end portion of the other hole pattern of the hole patterns, and a width of the half-tone region along a direction perpendicular to a line joining the two hole patterns being established in accordance with a distance between centers of the two hole patterns.

(Second Embodiment)

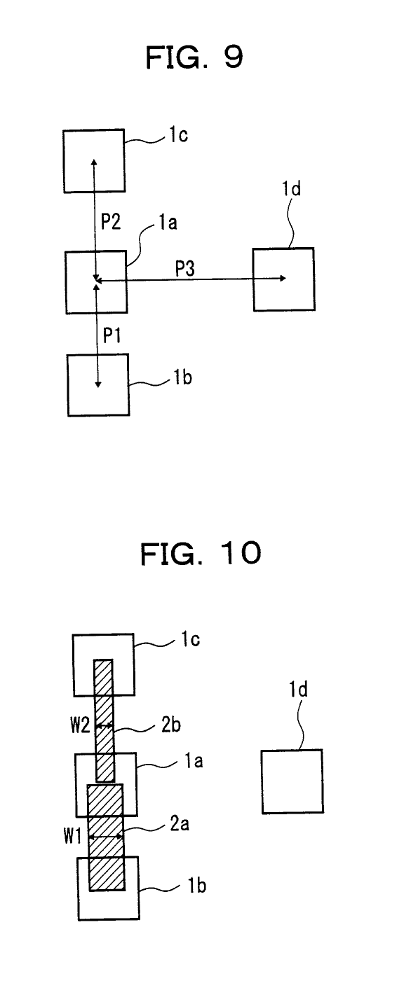

Next, consider the results of application of a half-tone phase-shift mask substrate having a standard transmissivity of 6%. The exposure conditions using KrF exima laser exposure system according to the second embodiment are same conditions as the first embodiment. The mask pattern before correction is shown in FIG. 9, which is a schematic representation showing a mask pattern of a photomask with no half-tone region according to a second comparison example. The hole dimensions in all cases is 0.18 μm, and the spacings of the hole pattern data between 1a to 1d are P1=0.3 μm, P2=0.34 μm, and P3=0.54 μm.

By disposing a half-tone region having a transmissivity of 6% between hole patterns having a narrow pitch, it is possible to prevent hole deformation. Under these conditions, the relationship between the pitch P of the hole pattern and the width W of the half-tone region is W=0.12 μm (for P=0.3 μm), W=0.8 μm (for P=0.31 to 0.34 μm), W=0.6 μm (for P=0.35 to 0.4 μm), and W=0.04 μm (for P=0.41 to 0.44 μm). The photomask after correction is shown in FIG. 10, which is a schematic representation showing the mask pattern of a photomask (phase-shift mask) according to the second embodiment of the present invention. The width W1 of the half-tone region 2a is 0.12 μm, and the width W2 of the half-tone 2b is 0.08 μm. Because the pitch between the hole patterns 1a and id is wide, no half-tone region is placed between these hole patterns.

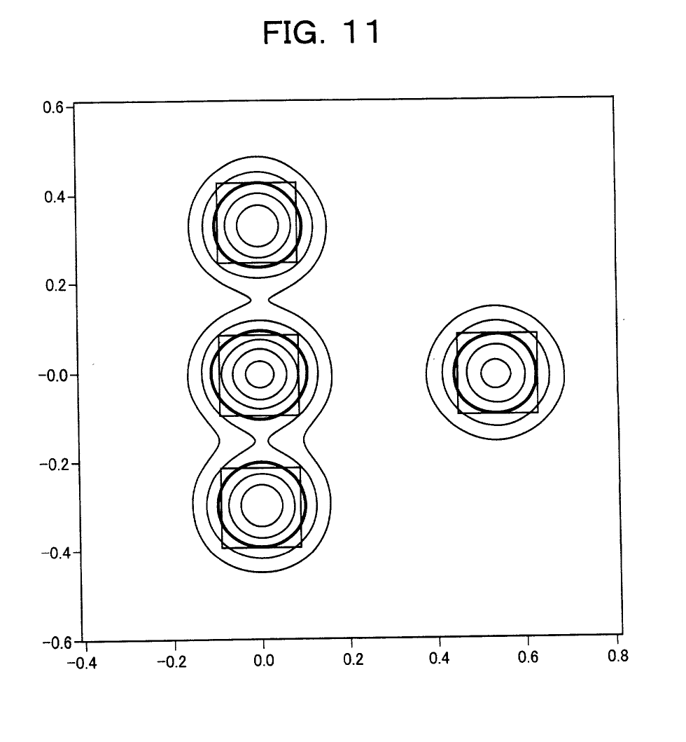

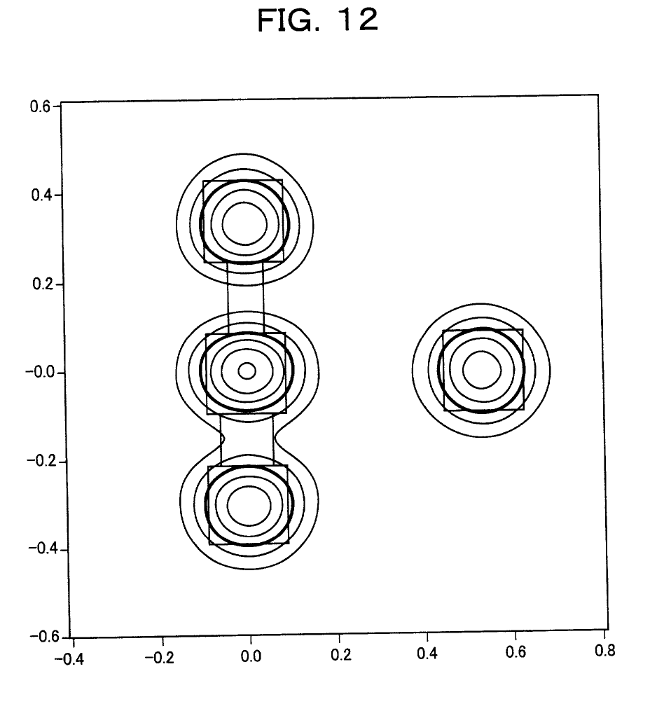

FIG. 11 and FIG. 12 are light intensity contour plots obtained by using mask patterns of FIG. 9 and FIG. 10, respectively. FIG. 11 is a schematic representation showing the light intensity contour plot of the light intensity distribution with a photomask having no half-tone region according to a second comparison example. FIG. 12 is a schematic representation showing the contour plot of the light intensity distribution for a photomask (phase shift mask) according to the second embodiment of the present invention. In particular the correction effect is most prominent between the hole patterns 11a and 11b, these having the minimum pitch. In FIG. 11 showing the condition before correction with the second comparison example, minimum pitch holes are joined together (because the light intensity between hole patterns is a high value exceeding 0.1, the photoresist is considerably developed). In contrast to this, in FIG. 12, which shows the second embodiment after correction, in the hole patterns of minimum pitch, sufficient reduction in the light intensity is achieved, and each hole pattern is formed separately.

Although it is possible to establish more precisely the width of the half-tone region, it is usually sufficient, such as shown in the above-described embodiment, to perform control with approximately four steps, over the range ±10 nm. If it is desired to perform even more precise control, it is necessary to set the pitch finer, and increase the number of steps of the width of the half-tone region.

(Third Embodiment)

A third embodiment of the present invention is described below.

Whereas in the first embodiment the application was for dimensional correction for a dense pattern with large-σ illumination or illumination at an inclination, in the third embodiment application is possible to prevention of deformation in a pattern that is dense in a single direction, for the case of small-σ illumination or medium-σ illumination (where the value of σ is 0.6 or smaller). In this case, a first case is that no correction is done to the region in which pattern is not dense. In a second case, if the pattern is dense in both the vertical and horizontal directions, (with a pitch less than a certain value), no correction is done. In a third case, if there is a hole pattern of high density in a single direction, a half-tone region having a width determined by the pitch of the pattern is applied. An additional fourth case is one intermediate between the above-noted second and third cases, that is, one in which in a pattern part such as in a dense array periphery, in which there are no other close patterns in one direction only (up, down, left, or right), a half-tone region having a width that is one-half that of the third case is applied.

The exposure conditions are the use of a KrF laser (248 nm), an NA of 0.68, and σ=0.5. With a hole dimension of 0.18 μm, in the case of arrangement in a single direction with a pitch of 0.32 μm, a half-tone region of 0.18 μm is used. The transmissivity of the half-tone region in this case is 3%.

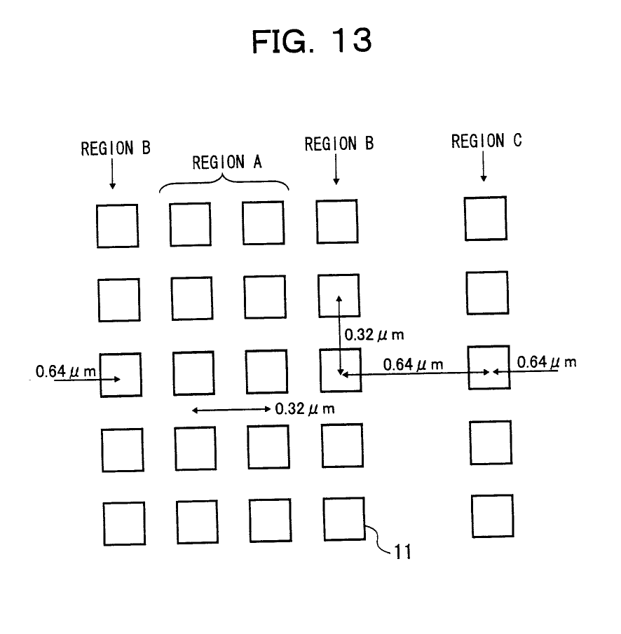

FIG. 13 shows a photomask with no half-tone pattern. FIG. 13 is a schematic representation showing a mask pattern of a photomask with no half-tone region according to the third comparison example. In FIG. 13, the hole patterns of 0.18 μm are all arranged in the vertical direction with a pitch of 0.32 μm. The part of the region A is a 0.32-μm pitch hole array region in which hole patterns are disposed to the left and right with a 0.32μm pitch. The regions B is the outer peripheral parts of the 0.32-μm pitch hole array of the region A. In one side of the regions B, there is no hole pattern, in the other side of the regions B, there is hole patterns of the region A with a pitch of 0.32 μm. The region C is a pattern with no other holes to either the left or right thereof (being at a distance from the region B holes).

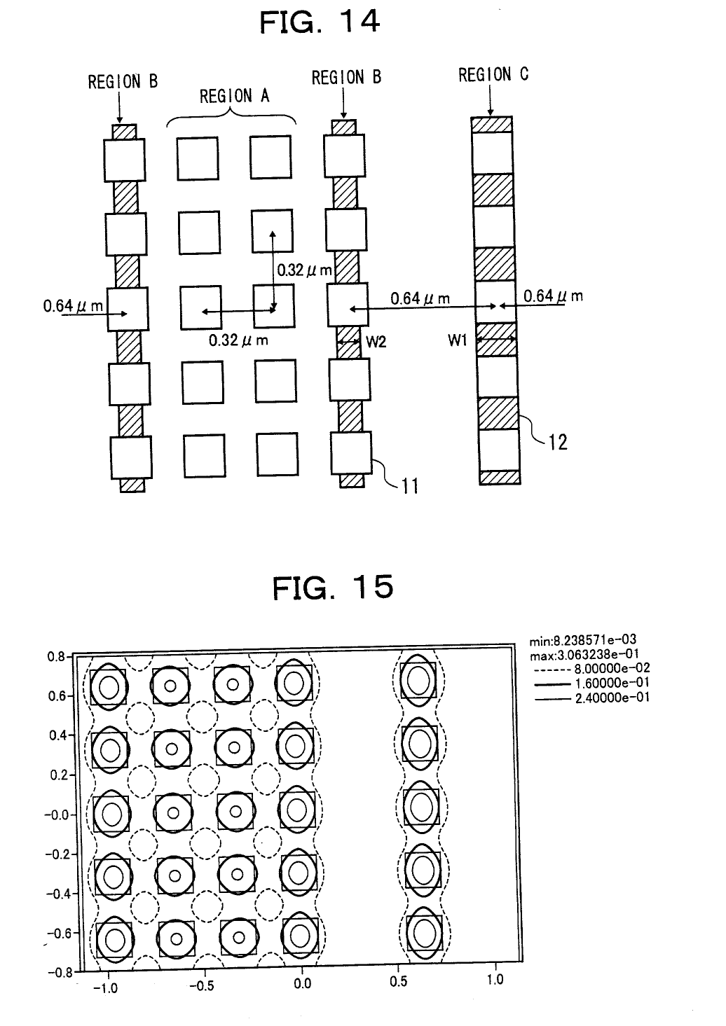

FIG. 14 shows the example of applying half-tone regions to this layout. FIG. 14 is a schematic representation showing the mask pattern of a photomask according to the third embodiment of the present invention. In the region A, no half-tone region is placed, in the region B a half-tone region with a width W2 of 0.09 μm is placed between each of the holes, and in the region C a half-tone region with a width W1 of 0.18 μm is placed between each of the holes.

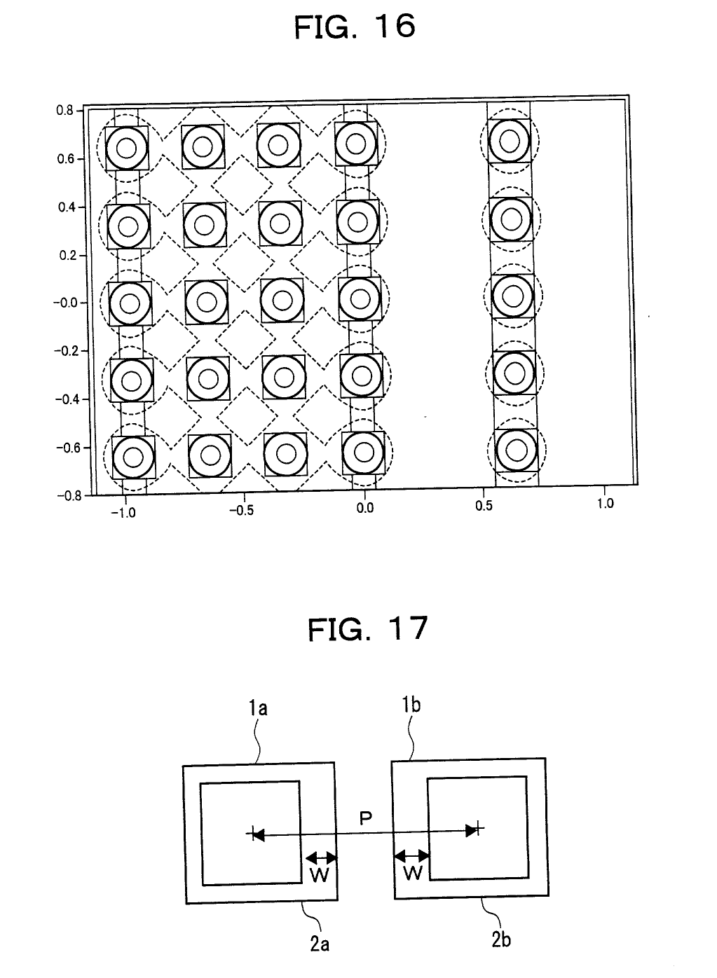

FIG. 15 shows the light intensity distribution according to the mask pattern shown in FIG. 13, and FIG. 16 shows the light intensity distribution according to the mask pattern shown in FIG. 14. FIG. 15 is a schematic representation showing a contour plot of the light intensity distribution of a photomask without a half-tone region according to the third comparison example, and FIG. 16 is a schematic representation showing a contour plot of the light intensity distribution of a photomask according to the third embodiment of the present invention. In these light intensity distributions, the light intensity of 0.16, which is the designed dimension for an independent hole of 0.18 μm is shown by a bold line, and the spacing between each of the contour lines is 0.08. In the light intensity distribution of FIG. 15 indicating the condition before correction, in the outer periphery of the dense array (region B) and in the part having a dense array in a single direction (region C), there is extension and joining of the hole patterns in the direction of density of arrangement. In the light intensity distribution of FIG. 16, which shows the condition after correction, it can be seen that the hole deformation has been prevented.

It is therefore possible to prevent deformation by investigating the neighboring patterns in two perpendicular directions, and by using a method of correction based on the pitch in these two directions, it is possible to prevent deformation at small and medium values of σ (σ<0.6).

(Fourth Embodiment)

A fourth embodiment of the present invention is described below, with reference to relevant drawings.

FIG. 17 is a schematic representation showing a mask pattern of a photomask according to the fourth embodiment. Whereas in the first embodiment (refer to FIG. 6) a single rectangular half-tone region is placed between hole patterns, in the fourth embodiment as shown in FIG. 17, the half-tone patterns 2a and 2b are provided in the peripheries of the hole patterns 1a and 1b.

The exposure conditions are the same as described for the first embodiment, these being the use of a KrF laser, a numerical aperture of 0.68, and a ⅔ annular illumination with σ of 0.75.

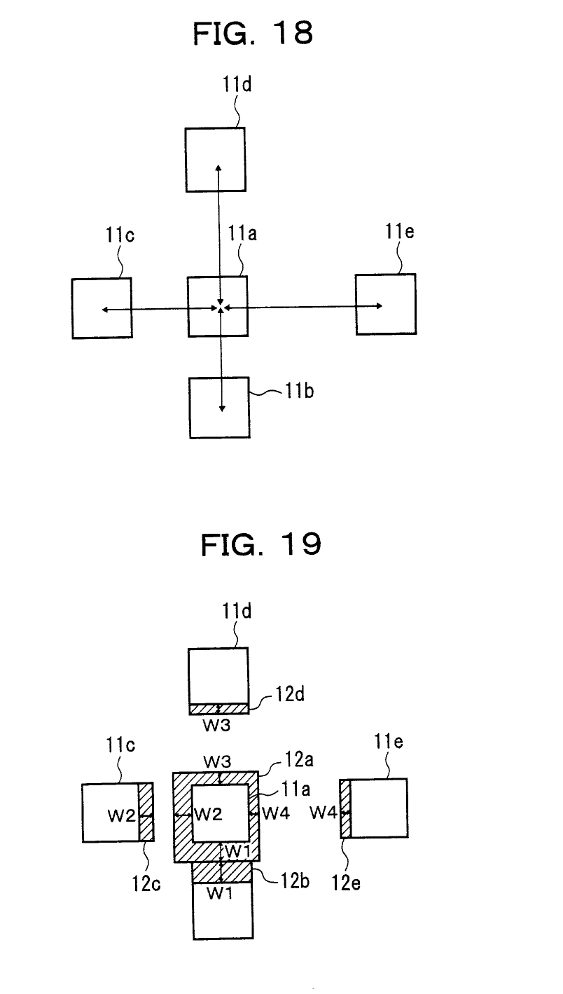

The result of the fourth embodiment is as follows. The photomask without a half tone region is shown in FIG. 18, which is a schematic representation showing a mask pattern according to the fourth comparison example. The hole diameter in this case is 0.18 μm, the spacing P1 between the hole patterns 11a and 11b is 0.3 μm, the spacing P2 between the hole patterns 11a and 11c is 0.34 μm, the spacing P3 between the hole patterns 11a and 11d is 0.42 μm, and the spacing P4 between the hole patterns 11a and lie is 0.5 μm. Under these exposure and pattern dimension conditions, the relationship of the pitch P to the half-tone region width W is W=0.06 μm (for P=0.3 μm), W=0.05 m (for P=0.31 to 0.38 μm), W=0.04 μm (for P=0.39 to 0.48 μm) and W=0.03 μm (for P=0.49 to 0.54 μm). FIG. 19 shows the mask pattern after correction, this FIG. 19 being a schematic representation showing the mask pattern of a photomask according to the fourth embodiment of the present invention, in which W1=0.06 μm, W2=0.05 μm, W3=0.04 μm, and W4=0.03 μm.

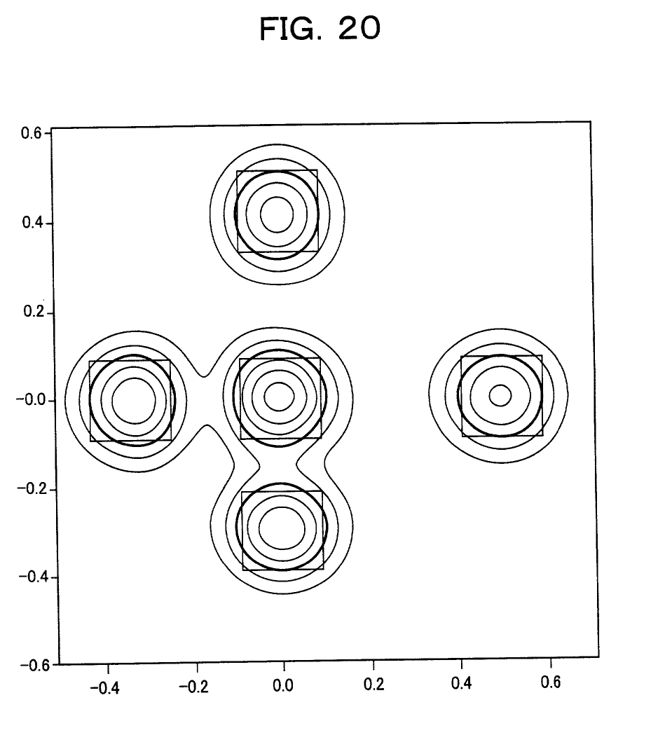

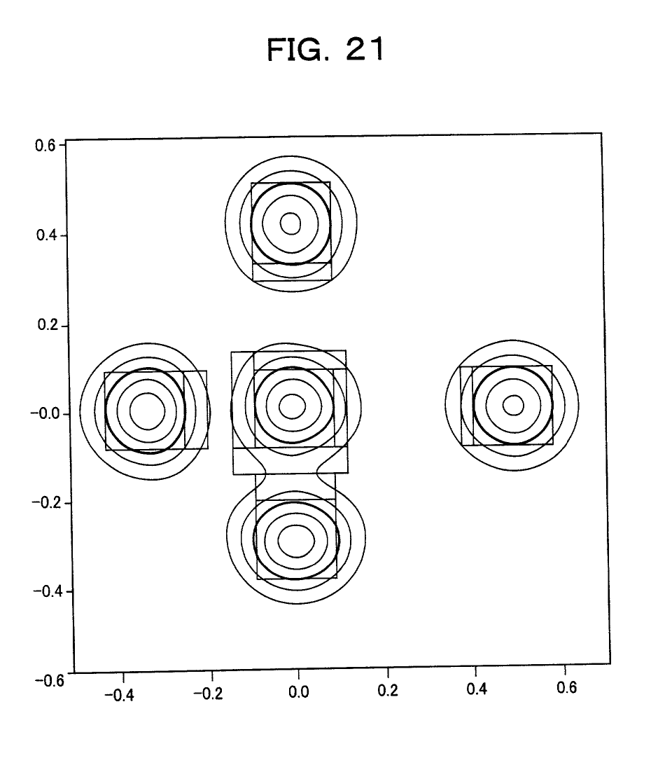

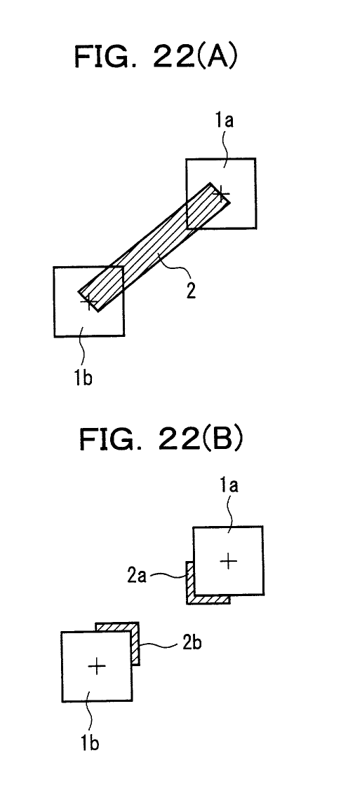

FIG. 20 shows the light intensity distribution of the mask pattern shown in FIG. 18, and FIG. 21 shows the light intensity distribution of the mask pattern shown in FIG. 19. FIG. 20 is a schematic representation showing a contour plot of the light intensity distribution for a photomask having no half-tone region according to the fourth comparison example, and FIG. 21 is a schematic representation showing a contour plot of the light intensity distribution for a photomask having a half-tone region according to the fourth embodiment of the present invention. In the results before correction, shown in FIG. 19, between the hole patterns 11a and 11b (pitch of 0.3 μm) there is joining of holes. In contrast to this, in the results after correction, shown in FIG. 20, it can be seen that joining of holes with a small pitch (between 11a and 11b) is prevented, with all holes formed with the desired dimensions.

It will be readily understood that the correction method of this embodiment can be applied to a so-called rim-type phase-shift mask, in which half-tone regions having the same width to all hole patterns are provided.

Additionally, although the foregoing embodiments were described for the case of a (transmission-type) photomask for a KrF exima laser, it will be understood that the present invention is not restricted in terms of exposure wavelength or mask structure, and that it can be applied in the same manner to a transmission-type mask having a membrane structure for X-ray exposure, or to a reflective-type mask having a multiplayer coating structure.

As described above, the fourth embodiment of the present invention is a photomask comprising: a substrate which either pass or reflect light, a half-tone film formed on the substrate and having at least two hole patterns which are disposed adjacently each other with a prescribed spacing, and a light-blocking film formed on the half-tone film and having hole patterns disposed in correspondence to the hole patterns of the half-tone film and having half-tone regions provided at an area between the two hole patterns, wherein one half-tone region of the half-tone regions is disposed continuously to an end portion of one hole pattern of the hole patterns and the other half-tone region of the half-tone regions is disposed continuously to an end portion of the other hole pattern of the hole patterns and the two half-tone regions are disposed opposite each other, and a width of the half-tone region along a direction of a line joining the two hole patterns being established in accordance with a distance between centers of the two hole patterns.

(Fifth Embodiment)

Additionally, application of the present invention is also possible in the case in which the neighboring hole pattern are arranged not vertically and horizontally, but rather along inclined directions.

The relationship between the pitch P, the width W of the half-tone region, and the hole dimension is as follows.

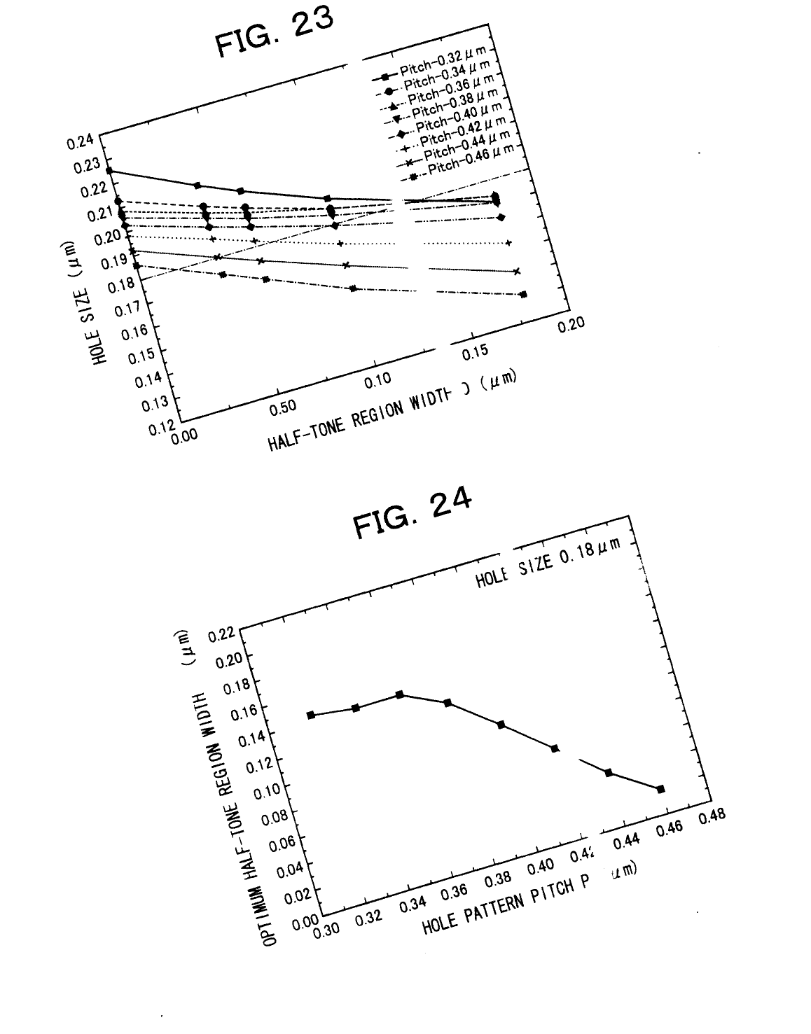

If the pitch is somewhat narrow (in this case P=0.32 to 0.46 μm) and fixed, the amount of reduction in the hole dimension can be seen to be proportional to the width W of the half-tone region and to the approximate ½-th power (square root) of the transmissivity T of the substrate. In principle, it is possible to understand this in that the intensity of the 0th order diffracted light, which is the average brightness is reduced in proportion to the width of the half-tone region and to the amplitude of the transmissivity (½-th power of the transmitted intensity). This is illustrated in FIG. 23, which is a graph showing the relationship between the half-tone region width W and the hole dimension for each of various pitches. The photomask used in this case being that of FIG. 2, corresponding to FIG. 4. It can be seen from this that the hole dimension decreases in proportion to the half-tone region width W. FIG. 24 is a graph that results from determining the optimum half-tone region width for each pitch P with a hole dimension of 0.18 μm. It can be seen from this that if the pitch P is within a certain range (in this case P=0.36 to 0.46 μm), there is a linear relationship with the half-tone region width W.

According to the present invention as described in detail above, even in the case of a photomask having a densely arranged hole pattern, it is possible by adding half-tone regions having a width in accordance with the hole pattern pitch, it is possible to prevent joining of pattern elements (holes) and improve the mask resolution.

Furthermore, it is possible to correct the dimensions and deformation in a fine hole pattern and in a fine pitch thereof.

What is claimed is:

1. A photomask comprising:
a substrate which either pass or reflect light, a half-tone film formed on said substrate and having at least two hole patterns which are disposed adjacently each other with a prescribed spacing, and
a light-blocking film formed on said half-tone film and having hole patterns disposed in correspondence to said hole patterns of said half-tone film and having a half-tone region provided at an area between said two hole patterns,
wherein said half-tone region is continuously disposed from an end portion of one hole pattern of said hole patterns to an end portion of the other hole pattern of said hole patterns, and a width of said half-tone region along a direction perpendicular to a line joining said two hole patterns being established in accordance with a distance between centers of said two hole patterns.

2. A photomask according to claim 1, wherein a width of said half-tone region become larger as said distance between said centers of two hole patterns becomes smaller.

3. A photomask according to claim 1, wherein an amount of reduction in a hole dimension of said hole pattern is proportional to said width of said half-tone region and to 1/n-th power of a transmissivity of said substrate.

4. A photomask according to claim 1, wherein, said half-tone region is provided if said distance between said centers of said two hole patterns is less than a prescribed length.

5. A photomask according to claim 1, wherein, when a plurality of said hole patterns are arranged with high density in one dimensional direction, said half-tone region is disposed on a line joining said plurality of hole patterns in said direction.

6. A photomask according to claim 1, wherein, when a plurality of said hole patterns are arranged with high density in two dimensional directions, said half-tone region is provided to a hole pattern disposed in an outer peripheral region of said plurality of hole patterns.

7. A photomask comprising:
a substrate which either pass or reflect light,
a half-tone film formed on said substrate and having at least two hole patterns which are disposed adjacently each other with a prescribed spacing, and
a light-blocking film formed on said half-tone film and having hole patterns disposed in correspondence to said hole patterns of said half-tone film and having half-tone regions provided at an area between said two hole patterns,
wherein one half-tone region of said half-tone regions is disposed continuously to an end portion of one hole pattern of said hole patterns and the other half-tone region of said half-tone regions is disposed continuously to an end portion of the other hole pattern of said hole patterns and said two half-tone regions are disposed opposite each other, and a width of said half-tone region along a direction of a line joining said two hole patterns being established in accordance with a distance between centers of said two hole patterns.

8. A photomask according to claim 7, wherein an amount of reduction in a hole dimension of said hole pattern is proportional to said width of said half-tone region and to 1/n-th power of a transmissivity of said substrate.

9. A photomask according to claim 7, wherein, said half-tone region is provided if said distance between said centers of said two hole patterns is less than a prescribed length.

10. A photomask according to claim 7, wherein, when a plurality of said hole patterns are arranged with high density in one dimensional direction, said half-tone region is disposed on a line joining said plurality of hole patterns in said direction.

11. A photomask according to claim 7, wherein, when a plurality of said hole patterns are arranged with high density in two dimensional directions, said half-tone region is provided to a hole pattern disposed in an outer peripheral region of said plurality of hole patterns.
